(19) United States
(12) Patent Application Publication
(10) Pub. No.: US 2020/0290069 A1
(43) Pub. Date: Sep. 17, 2020

(54) PORTABLE DEVICE FOR SPREADING ODORS

(71) Applicant: Whitescent Ltd., Netanya (IL)

(72) Inventor: Lior THAU, Kfar Vitkin (IL)

(73) Assignee: Whitescent Ltd., Netanya (IL)

(21) Appl. No.: 16/439,760

(22) Filed: Jun. 13, 2019

(30) Foreign Application Priority Data
Mar. 17, 2019 (IL) 265413

(51) Int. Cl. B05B 7/24; B05B 12/00

(52) U.S. Cl. CPC B05B 7/2464; B05B 12/002

(57) ABSTRACT

A portable device for spreading odors is disclosed. The portable device may include: a housing configured to hold: at least one replaceable cartridge holding an absorbing element absorbed with a volatile liquid; and a ventilation unit configured to provide air flow to the vicinity of the at least one absorbing element. The hosing may include: at least one insertion entrance for inserting the at least one replaceable cartridge; and at least one volatile compound exit.

### CROSS REFERENCE TO RELATED APPLICATIONS

This application claims the benefit of Israeli Patent Application Serial No. 265413, filed on Mar. 17, 2019 and entitled PORTABLE DEVICE FOR SPREADING ODORS, which is incorporated in its entirety herein by reference.

### TECHNICAL FIELD OF THE INVENTION

The present invention generally relates to devices for spreading odors. More particularly, the present invention relates to portable devices for spreading odors.

### BACKGROUND OF THE INVENTION

Humans are very sensitive to odors and odors are known to impact ones' feelings. Perfumes delights us, smells of food make us hungry, the smell of another person's sweat may repulse us and the like. While, one can control the environment's smell at one's home or office, it is almost impossible at public places (e.g., the subway, a bus, an airplane, public toilets and the like) or at other people's places.

The current solutions include air sprays that spread a single amount of aerosol from a pressurized tank or a bottle filled with liquid perfume. Such solutions are limited to a single act of spreading of odors each time and are considered unsafe (e.g., can explode, are flammable, etc.) and for example, cannot be carried on board a passengers' compartment in an airplane.

Accordingly, there is a need for a simple, lightweight and safe solution for ongoing spreading of odors in the vicinity of a user. Such solutions may allow the user to select the radius at which the odors are to be spread.

### SUMMARY OF THE INVENTION

Some aspects of the invention may be related to a portable device for spreading odors, that may include: a housing configured to hold: at least one replaceable cartridge holding an absorbing element absorbed with a volatile liquid; and a ventilation unit configured to provide air flow to the vicinity of the at least one absorbing element. In some embodiments, the hosing may include: at least one insertion entrance for inserting the at least one replaceable cartridge; and at least one volatile compound exit.

In some embodiments, the replaceable cartridge may include one or more apertures configured to introduce the provided air flow to the vicinity of the at least one absorbing element. In some embodiments, the replaceable cartridge may include one or more exits directed to release volatile compounds evaporated from the absorbing element. In some embodiments, the ventilation unit may include at least one fan.

In some embodiments, the portable device may further include a controller configured to control the air flow provided by the ventilation unit. In some embodiments, the controller may be configured to control the amount of power provided to a fan. In some embodiments, the controller may further include a user interface. In some embodiments, the controller may be configured to: receive instructions from the user interface; and control the capacity of air flow provided by the ventilation unit based on the received instructions. In some embodiments, the user interface comprises at least one of: a button, a key, a microphone and a touchscreen.

In some embodiments, the ventilation unit may be configured to allow the volatile compound to expend in a radius of at least 5 meters. In some embodiments, the controller may be configured to: receive a required radius spread of the volatile compound; and control the ventilation unit to provide an air flow in a capacity that causes the required radius spread.

In some embodiments, the device may further include a communication unit configured to communicate with an external device. In some embodiments, the controller may further be configured to: receive a capacity of air flow from an application running on the external device via the communication unit; and control the air flow provided by the ventilation unit based on the received capacity.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A and 1B are illustrations of portable devices for spreading odors according to some embodiments of the invention;

FIG. 2 is an illustration of an air flow in a portable device for spreading odors according to some embodiments of the invention;

FIG. 3 is a block diagram of a portable devices for spreading odors according to some embodiments of the invention; and

FIG. 4 is an illustration of a set of portable devices for spreading odors and cartridges according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

A portable device for spreading odors according to embodiments of the invention may be carried, e.g., manually, by a human, in a bag (e.g., a school bag, a shoulder bag and the like) to be used anywhere. In some embodiments, the liquid odor material included in such portable device may be stored in a way that may prevent the liquid odor from spilling out from the portable device, regardless of its position and orientation (e.g., inside a bag). The device may be easily carried and activated single handedly. A user may activate the device in any environment, either closed environment (e.g., a bus, passengers' compartment at an airplane, public toilets and the like), an open environment (e.g., in a picnic) or a semi closed environment (e.g., passenger's hall in a central station, at a train station platform and the like). In some embodiments, such a device may include a replaceable cartridge for providing the odors and a ventilation unit that allows to spread the odors in the vicinity of the user. In some embodiments, the ventilation unit may allow the odors to be sprayed in a radius of several meters (e.g., 5 meters).

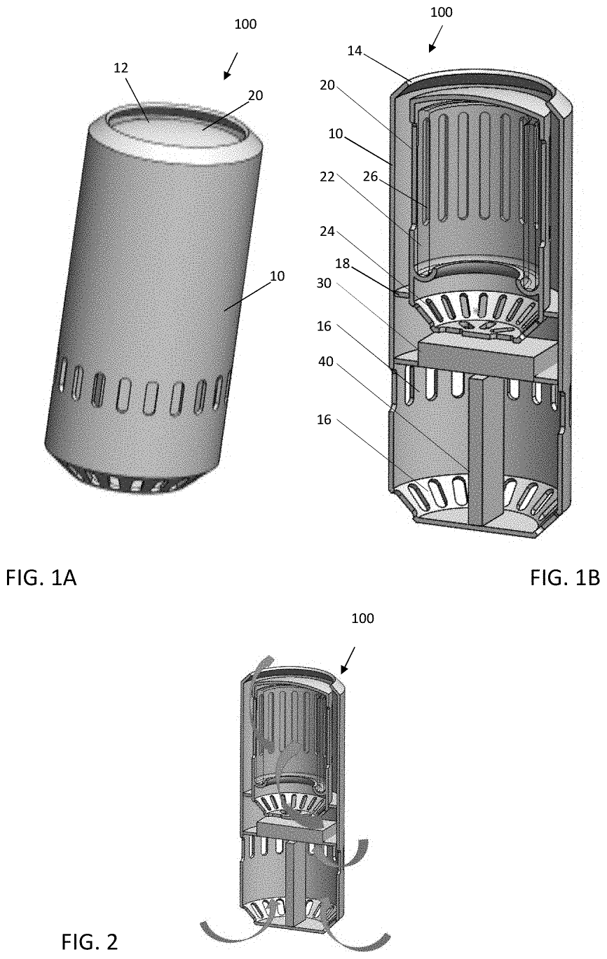

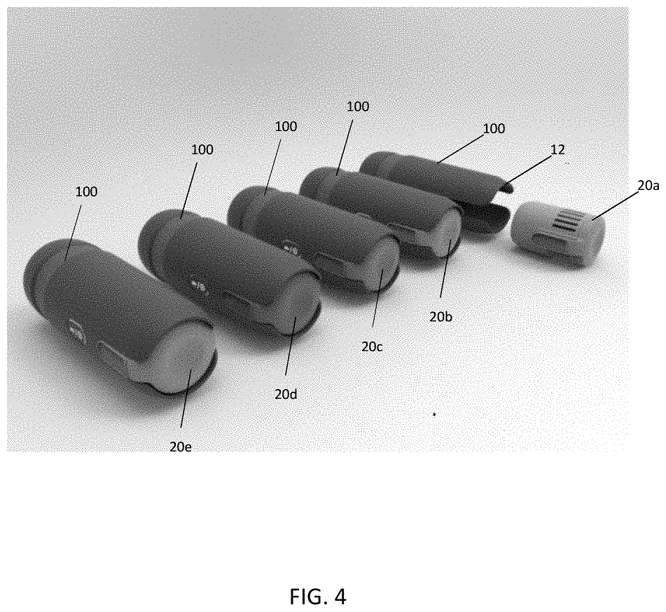

Reference is now made to FIGS. 1A and 1B which are illustrations of a portable device 100 for spreading odors according to some embodiments of the invention. FIG. 1B is a cross-section view of the device illustrated in FIG. 1A. Portable device 100 may include: a housing 10, at least one replaceable cartridge 20 holding an absorbing element 22 absorbed with a volatile liquid (e.g., liquid odor) and a ventilation unit 30 configured to provide air flow to the vicinity of at least one absorbing element 22. In some embodiments, housing 10 may include at least one insertion entrance 12 for inserting at least one replaceable cartridge 20. For example, replaceable cartridge 20 may be inserted from insertion entrance 12, as illustrated in FIG. 4. In yet another example (not illustrated), hosing 10 may be configured to be separated from a frame 18 holding at least one replaceable cartridge 20, via insertion entrance 12, thus allowing the replacement of at least one replaceable cartridge 20 with a different one. In some embodiments, hosing 10 may include at least one volatile compound (VOC) exit 14 configured to allow VOCs evaporated from absorbing element 22 to exit and be spread in the surrounding of device 100. In some embodiments, hosing 10 may include one or more air entrances 16 configured to allow air from the surrounding of device 100 to enter and be ventilated trough cartridge 20 as illustrated and explained with respect to FIG. 2.

In some embodiments, hosing 10 may be made from any suitable material, for example, a polymer, a metal (e.g., an aluminum alloy), wood and a combination thereof.

In some embodiments, absorbing element 22 may include any absorbing material configured to absorb volatile liquid. In some embodiments, absorbing material may include a fabric (e.g., cotton), a porous polymer or any other of porous material and foams. For example, absorbing element 22 may include at least one of: a felt having a thickness of 1-50 mm., a cardboard at various thickness, aroma beads, polymeric foam infused with aromatic volatile liquid, ceramic foam infused with aromatic volatile liquid and the like. In some embodiments, the volatile liquid may include VOCs configured to spread odors (e.g., perfumes), odors neutralizers and the like. For example, aromatic oils, liquid perfumes, pest control material (e.g., Citronella oil) and the like.

In some embodiments, replaceable cartridge 20 may be replaced by another replaceable cartridge 20 and a user may select a replaceable cartridge 20 from a variety of replaceable cartridges 20 each carrying a different volatile liquid for spreading different odors. In some embodiments, the user may carry, for example, in his/her bag, several different replaceable cartridges 20 and may replace them as needed. For example, the user may use one type of replaceable cartridge 20 in public toilets and another type in his/her office using the same device 100.

In some embodiments, replaceable cartridge 20 may include one or more apertures 24 configured to introduce flow of air provided by ventilation system 30 to the vicinity of at least one absorbing element 22. In some embodiments, replaceable cartridge 20 may include one or more exits 26 directed to release the VOCs evaporated from absorbing element 22. In some embodiments, apertures 24 and exits 26 may be located in the VOCs air flow path, as illustrated in FIG. 2.

In some embodiments, ventilation unit 30 may include any component that may allow to controllably introduce air from the surrounding of device 100 to the vicinity of absorbing element 22 and further from absorbing element 22 towards VOCs exit 14. For example, ventilation unit 30 may include a fan.

In some embodiments, device 100 may further include a power supply source 40 that may be a battery (e.g., a rechargeable battery). For example, device 100 may include a socket for recharging power supply source 40 (e.g., using a USB cable).

Reference is now made to FIG. 2 which is an illustration of the air flow into and from device 100 during operation of device 100. Air may flow from the surroundings of device 100 via one or more entrances 16. Entrances 16 may include slots located at the any end and/or walls of housing 10. In some embodiments, since the volatile liquid is totally absorbed in absorbing element, therefore may not spill out from device 100, one or more entrances 16 may be located at any position at the end and/or walls of housing 10, regardless of the position of at least one replaceable cartridge 20. From the entrances the air may be pumped and directed towards apertures 24 of cartridge 20 to be directed to the vicinity of absorbing element 22. In some embodiments, at the vicinity of absorbing element 22 the air may absorb VOCs evaporated from absorbing element 22 to be carried by the air flow from exit 14 to the surrounding of device 100. In some embodiments, device 100 may be configured to spread the air carrying VOCs in a radius of at most 5 meters in an open or semi-open environment. In some embodiments, the spreading radius may be controlled by controlling the amount of air provided by ventilation system 30, as discussed herein below. In some embodiments, device 100 may be configured to spread odors in 40-50 m² closed rooms.

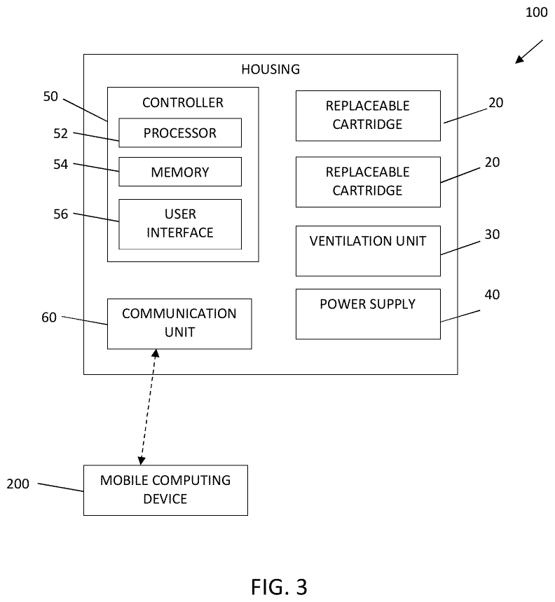

In some embodiments, device 100 may include a controller configured to control various controllable components of device 100, illustrated and discussed in FIG. 3.

Reference is now made to FIG. 3 which is a block diagram of device 100 according to embodiments of the invention. Device 100 may include a controller 50 configured to control various controllable components of device 100, for example, the power provided from power supply 40 to ventilation system 30. Controller 100 may include a processor 52, a memory 54 and a user interface. In some embodiments, processor 52 may be any computing device configured to execute instructions stored in memory 54. In some embodiments, user interface 56 may include any device that may be configured to send, receive and/or present information to or from the processor/controller, and/or to a user. For example, user interface 56 may include at least one of: one or more light emitting diodes (LEDs), one or more push buttons, a touchscreen, a knob, a microphone, a speaker and the like.

In some embodiments, a user may select the capacity of air and VOCs provided by device 100, by pushing a button, rotating a knob, touching a touchscreen and the like. Following the selection, controller 50 may control the amount of power provided by power supply 40 to ventilation unit 30, such that device 100 may provide the selected capacity. For example, controller 50 may control the amount of power provided to the one or more fans.

In some embodiments, device 100 may further include a communication unit 60 configured to communicate with an external device, for example, a mobile device of the user. For example, communication unit 60 may include at least one cell modem 2G/3G/CAT-M, Bluetooth device or any other wireless modem. In some embodiments, controller 50 may receive the selected VOCs capacity (e.g., air flow capacity) from an application running on the external device, for example, the user's mobile computing device 200 (e.g., a smart phone, a smart watch, a lap top and the like) being in communication with portable device 100 via communication unit 60.

In some embodiments, power supply 40, controller 50 and communication unit 60 may all be assembled on a printed circuit board (PCB). In some embodiments, the fan of ventilation unit 30 may also be assembled on the same PCB or elsewhere.

Reference is now made to FIG. 4 which is an illustration of several devices 100 for spreading odors and replaceable cartridges 20 according to some embodiments of the invention. As illustrated in FIG. 4 each of devices 100 may include any one of cartridges 20a-20e. In some embodiments, at least some of cartridges 20a-20e may include different odors (e.g., indicated as different colors). In some embodiments, cartridges 20a-20e may be inserted into devices 100 via insertion entrance 12.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A portable device for spreading odors, comprising:
a housing configured to hold:
at least one replaceable cartridge holding an absorbing element absorbed with a volatile liquid; and
a ventilation unit configured to provide air flow to the vicinity of the at least one absorbing element,
wherein the hosing comprises
at least one insertion entrance for inserting the at least one replaceable cartridge; and
at least one volatile compound exit.

2. The portable device of claim 1, wherein the replaceable cartridge comprises one or more apertures configured to introduce the provided air flow to the vicinity of the at least one absorbing element.

3. The portable device of claim 1, wherein the replaceable cartridge comprises one or more exits directed to release volatile compounds evaporated from the absorbing element.

4. The portable device of claim 1, wherein the ventilation unit comprises at least one fan.

5. The portable device of claim 1, further comprising a controller configured to control the air flow provided by the ventilation unit.

6. The portable device of claim 5, wherein the controller is configured to control the amount of power provided to a fan.

7. The portable device of claim 5, wherein the controller further comprises a user interface.

8. The portable device of claim 7, wherein the controller is configured to:
receive instructions from the user interface; and
control a capacity of air flow provided by the ventilation unit based on the received instructions.

9. The portable device of claim 8, wherein the user interface comprises at least one of: a button, a key, a microphone and a touchscreen.

10. The portable device of claim 1, wherein the ventilation unit is configured to allow the volatile compound to expend in a radius of at least 5 meters.

11. The portable device of claim 5, wherein the controller is configured to:
receive a required radius spread of the volatile compound; and
control the ventilation unit to provide an air flow in a capacity that causes the required radius spread.

12. The portable device of claim 5, further comprising a communication unit configured to communicate with an external device.

13. The portable device of claim 12, wherein the controller is further configured to:
receive a capacity from an application running on the external device via the communication unit; and
control the air flow provided by the ventilation unit based on the received capacity.

14. The portable device of claim 6, wherein the controller further comprises a user interface.

15. The portable device of claim 7, wherein the user interface comprises at least one of: a button, a key, a microphone and a touchscreen.

16. The portable device of claim 14, wherein the user interface comprises at least one of: a button, a key, a microphone and a touchscreen

17. The portable device of claim 14, wherein the controller is configured to:
receive instructions from the user interface; and
control a capacity of air flow provided by the ventilation unit based on the received instructions.
